(19) United States
(12) Patent Application Publication
(10) Pub. No.: US 2012/0130481 A1
(43) Pub. Date: May 24, 2012

(54) LOCAL VASCULAR DELIVERY OF ADENOSINE A2A RECEPTOR AGONISTS IN COMBINATION WITH OTHER AGENTS TO REDUCE MYOCARDIAL INJURY

(76) Inventors: Robert Falotico, Belle Mead, NJ (US); Andrew Luk, Pleasanton, CA (US); Theodore L. Parker, Danville, CA (US); Jonathon Z. Zhao, Belle Mead, NJ (US)

(21) Appl. No.: 12/971,083

(22) Filed: Dec. 17, 2010

Related U.S. Application Data
(60) Provisional application No. 61/415,056, filed on Nov. 18, 2010.

(51) Int. Cl. A61F 2/82

(52) U.S. Cl. 623/1.42

(57) ABSTRACT

A stent or other implantable medical device for the local delivery of a selective adenosine receptor agonist may be utilized in combination with other therapeutic agents to reduce myocardial injury following an acute myocardial infarction. As soon as possible following an acute myocardial infarction a stent or other suitable device comprising and capable of delivering a selective adenosine receptor agonist is positioned in the blood vessel with the occlusion responsible for causing the infarct. Once in position , the stent or other intraluminal device is deployed to remove the occlusion and reestablish blood flow to the specific area, region or tissue volume of the heart. Over a given period of time the selective adenosine receptor agonist alone or in combination with other therapeutic agents elute from the stent or other device into the downstream coronary blood flow into the hypoxic cardiac tissue for a time sufficient to reduce the level of myocardial injury.

### CROSS REFERENCE TO RELATED APPLICATIONS

This application claims the benefit of U.S. Provisional Patent Application Ser. No. 61/415,056 filed Nov. 18, 2010.

### BACKGROUND OF THE INVENTION

1. Field of the Invention

The present invention relates to the local administration of therapeutic agents and/or therapeutic agent combinations for reducing myocardial injury following an acute myocardial infarction, and more particularly to intraluminal medical devices for the local delivery of therapeutic agents and/or therapeutic agent combinations for reestablishing perfusion and reducing myocardial injury following an acute myocardial infarction.

2. Discussion of the Related Art

Many individuals suffer from circulatory or vascular disease caused by a progressive blockage or narrowing of the blood vessels that perfuse the heart and other major organs. More severe blockage of blood vessels in such individuals often leads to hypertension, ischemic injury, stroke, or myocardial infarction. Atherosclerotic lesions, which limit or obstruct coronary blood flow, are the major cause of ischemic heart disease. Alternatively, spontaneous rupture of inflammatory atherosclerotic lesions or vulnerable plaque may lead to intermittent or complete thrombotic occlusion of an artery causing ischemic injury such as stroke and/or acute myocardial infarction. Percutaneous transluminal coronary angioplasty is a medical procedure for which the purpose is to increase blood flow through an artery. Percutaneous transluminal coronary angioplasty is the predominant treatment for coronary artery stenosis. The increasing use of this procedure is attributable to its relatively high success rate and its minimal invasiveness compared with coronary bypass surgery. A limitation associated with percutaneous transluminal coronary angioplasty is the abrupt closure of the vessel, which may occur immediately after the procedure and restenosis, which occurs gradually following the procedure. Additionally, restenosis is a chronic problem in patients who have undergone saphenous vein bypass grafting. The mechanism of acute occlusion appears to involve several factors and may result from vascular recoil with resultant closure of the artery and/or deposition of blood platelets and fibrin along the damaged length of the newly opened blood vessel.

Restenosis after percutaneous transluminal coronary angioplasty is a more gradual process initiated by vascular injury. Multiple processes, including thrombosis, inflammation, growth factor and cytokine release, cell proliferation, cell migration and extracellular matrix synthesis each contribute to the restenotic process.

Upon pressure expansion of an intracoronary balloon catheter during angioplasty, smooth muscle cells and endothelial cells within the vessel wall become injured, initiating a thrombotic and inflammatory response. Cell derived growth factors such as platelet derived growth factor, basic fibroblast growth factor, epidermal growth factor, thrombin, etc., released from platelets, invading macrophages and/or leukocytes, or directly from the smooth muscle cells provoke a proliferative and migratory response in medial smooth muscle cells. These cells undergo a change from the contractile phenotype to a synthetic phenotype characterized by only a few contractile filament bundles, extensive rough endoplasmic reticulum, Golgi and free ribosomes. Proliferation/migration usually begins within one to two days' post-injury and peaks several days thereafter (Campbell and Campbell, 1987; Clowes and Schwartz, 1985).

Daughter cells migrate to the intimal layer of arterial smooth muscle and continue to proliferate and secrete significant amounts of extracellular matrix proteins. Proliferation, migration and extracellular matrix synthesis continue until the damaged endothelial layer is repaired at which time proliferation slows within the intima, usually within seven to fourteen days post-injury. The newly formed tissue is called neointima. The further vascular narrowing that occurs over the next three to six months is due primarily to negative or constrictive remodeling.

Simultaneous with local proliferation and migration, inflammatory cells adhere to the site of vascular injury. Within three to seven days post-injury, inflammatory cells have migrated to the deeper layers of the vessel wall. In animal models employing either balloon injury or stent implantation, inflammatory cells may persist at the site of vascular injury for at least thirty days (Tanaka et al., 1993; Edelman et al., 1998). Inflammatory cells therefore are present and may contribute to both the acute and chronic phases of restenosis.

Unlike systemic pharmacologic therapy, stents have proven useful in significantly reducing restenosis. Typically, stents are balloon-expandable slotted metal tubes (usually, but not limited to, stainless steel or cobalt-chromium alloys, which, when expanded within the lumen of an angioplastied coronary artery, provide structural support through rigid scaffolding to the arterial wall. This support is helpful in maintaining vessel lumen patency. In two randomized clinical trials, stents increased angiographic success after percutaneous transluminal coronary angioplasty, by increasing minimal lumen diameter and reducing, but not eliminating, the incidence of restenosis at six months (Serruys et al., 1994; Fischman et al., 1994). In addition, stents have become the treatment of choice for revascularization of a thrombosed coronary artery (acute myocardial infarction) in which rapid restoration of blood flow to ischemic myocardial tissue is the primary determinant of long term clinical benefit . Full restoration of coronary blood flow with a stent within 6 hours of presentation of symptoms, and preferably under 3 hours, has been shown to produce superior clinical outcomes over administration of a thrombolytic agent (tPA, streptokinase, etc.) to dissolve a thrombotic occlusion.

Stents utilized for the local delivery of rapamycins, including sirolimus, everolimus and other rapamycin analogs and derivatives (mTOR inhibitors), have proved more successful in significantly reducing restenosis and related complications following percutaneous transluminal angioplasty and other similar arterial/venous procedures than bare metal stents. Rapamycins may be incorporated onto or affixed to the stent in a number of ways. For example, the rapamycins may be incorporated into a polymeric matrix and then affixed to the surface of the stent by any suitable means. Alternately, the rapamycins may be incorporated into a polymeric matrix and then loaded into reservoirs on or in the stent. Either way, the rapamycins elute from the polymeric matrix over a given period of time and into the surrounding tissue.

Additionally, heparin coating of stents appears to have the added benefit of producing a reduction in sub-acute thrombosis after stent implantation. Thus, sustained mechanical expansion of a stenosed coronary artery with a stent has been shown to provide some measure of restenosis prevention, and the coating of stents with rapamycins and heparin has demonstrated both the feasibility and the clinical usefulness of delivering drugs locally, at the site of injured tissue.

Given that the feasibility and the desirability of local delivery of drugs via a stent has been demonstrated, stents as well as other implantable medical devices may be utilized to deliver other drugs or therapeutic agents to arteries as well as organs downstream of the placement of the stent or other medical device to treat other conditions. For example, there exists a need for the local delivery of agents for reducing myocardial injury following an acute myocardial infarction. More generally, there exists a need for the local administration of therapeutics to reduce ischemic injury. In addition, combinations of therapeutic agents may be locally delivered to treat other potentially related as well as other non-related complications and conditions.

### SUMMARY OF THE INVENTION

The local delivery, via a stent or other suitable device, of a selective adenosine receptor agonist in accordance with the present invention may be utilized to overcome the drawbacks of treatments set forth above.

In accordance with one aspect, the present invention is directed to a medical device for the local delivery of a selective adenosine receptor agonist in combination with at least one additional therapeutic agent for the treatment of myocardial injury following an acute myocardial infarction. The medical device comprising an expandable intraluminal device configured for opening and reestablishing blood flow in a vessel at least partially occluded, a selective adenosine receptor agonist releasably affixed to the expandable intraluminal device, the selective adenosine receptor agonist being configured to elute into the bloodstream at a rate of at least ten micrograms per hour for at least four (4) hours after the reestablishment of blood flow in the vessel, and a phosphodiesterase inhibitor affixed to the expandable intraluminal device and configured to elute into at least one of the bloodstream and the surrounding tissue.

In accordance with another aspect, the present invention is directed to a method for treating myocardial injury following an acute myocardial infarction. The method comprising expanding an intraluminal device to open and reestablish blood flow in a vessel at least partially occluded, releasing a selective adenosine receptor agonist from the expandable intraluminal device into the bloodstream at a rate of at least ten micrograms per hour for at least four (4) hours after the reestablishment of blood flow in the vessel, and releasing a phosphodiesterase inhibitor from the expandable intraluminal device into at least one of the bloodstream and the surrounding tissue.

A stent or other implantable medical device for the local delivery of an adenosine A_{2A} receptor agonist may be utilized to reduce myocardial injury following an acute myocardial infarction. As soon as possible following an acute myocardial infarction, a stent or other suitable device comprising and capable of delivering an adenosine A_{2A} receptor agonist is positioned in the blood vessel with the occlusion responsible for causing the infarct. Once in position, the stent or other intraluminal device is deployed to remove the occlusion and reestablish blood flow to the specific area, region or tissue volume of the heart. Over a given period of time described in detail subsequently, the adenosine A_{2A} receptor agonist elutes from the stent or other device into the downstream coronary blood flow into the hypoxic cardiac tissue for a time sufficient to reduce the level of myocardial injury. As described herein, the present invention may also be utilized to treat other organs.

The early and sustained release of the adenosine A_{2A} receptor agonist may reduce myocardial injury be reducing the size or amount of infarcted myocardial tissue, reducing the level of myocellular death, reduce the extent of reperfusion injury, preserve more function in the myocapillary bed and or mitigate the so-called “no-reflow” condition. These effects should, in turn, improve cardiac output, ejection fraction and cardiac wall motion post infarct. The delivery of the adenosine A_{2A} receptor agonist from the stent or other device to the hypoxic tissue will begin immediately after the occluded vessel has been made patent by deployment of the device, or more specifically, the delivery of the agent from the device will not begin until blood flow is reestablished to the treatment site as the blood carries the therapeutic agent downstream. In the case of a surface coated drug eluting stent or reservoir eluting stent, delivery of the adenosine A_{2A} receptor agonist will begin immediately upon expansion of the stent and removal of the balloon which will allow the agonist to elute. If a self expanding stent is utilized, agonist delivery will begin upon deployment of the stent and contact with the blood.

In addition, the combination of sirolimus and cilostazol may be more efficacious than either drug alone in reducing both smooth muscle cell proliferation and migration. Cilostazol may also be utilized to achieve prolonged anti-platelet deposition and prevention of thrombosis on the stent or other medical device. In addition, cilostazol which is a PDE-III inhibitor also functions similarly to the selected adenosine A_{2A} receptor agonists described herein. Phosphodiesterase inhibitors (PDEi) act by blocking or retarding the enzymatic conversion of cyclic-adenosine monophosphate (c-AMP) to 5′-adenosine monophosphate (AMP). Adenosine A_{2A} receptors are Gₛ protein coupled receptors that are linked to activation of adenyl cyclase. Adenosine A_{2A} receptor agonist can therefore elevate intracellular cAMP levels in target cells to exert their biological effects. Since cAMP levels in tissue are directly reflective of adenosine receptor stimulation, blocking degradation of c-AMP with a PDEi will result in a greater stimulation of the same adenosine receptors as those targeted by exogenous adenosine receptor agonists. In that sense, the combination of a PDEi and a selective adenosine receptor agonist delivered together will provide an enhanced level of stimulation of adenosine receptors compared to either class given alone. Accordingly, the combination of drugs may be utilized to treat restenosis, thrombosis, and to reduce myocardial injury following an acute myocardial infarction when delivered from a stent or other medical device.

Local delivery may be utilized in combination with systemic delivery of the same and/or different therapeutic agents.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 is a view along the length of a stent (ends not shown) prior to expansion showing the exterior surface of the stent and the characteristic banding pattern.

FIG. 2 is a perspective view along the length of the stent of FIG. 1 having reservoirs in accordance with the present invention.

FIG. 3 is an isometric view of an expandable medical device with a beneficial agent loaded in holes in accordance with the present invention.

FIG. 4 is an enlarged side view of a portion of an expandable medical device with beneficial agent openings in the bridging elements in accordance with the present invention.

FIG. 5 is a diagrammatic, side view representation of a portion of a drug eluting stent in accordance with the present invention.

FIG. 6 is a graphical representation of coronary blood flow in anesthetized, open-chest pigs with implanted bare metal stents and implanted stents eluting ATL-359 in accordance with the present invention.

FIG. 7 is a diagrammatic representation of a first exemplary embodiment of a stent coated with a combination of sirolimus and cilostazol in accordance with the present invention.

FIG. 8 is a graphical representation of the in vitro release kinetics of a first exemplary sirolimus and cilostazol combination stent coating in accordance with the present invention.

FIG. 9 is a diagrammatic representation of a second exemplary embodiment of a stent coated with a combination of sirolimus and cilostazol in accordance with the present invention.

FIG. 10 is a graphical representation of the in vitro release kinetics of a second exemplary sirolimus and cilostazol combination stent coating in accordance with the present invention.

FIG. 11 is a diagrammatic representation of a third exemplary embodiment of a stent coated with a combination of sirolimus and cilostazol in accordance with the present invention.

FIG. 12 is a graphical representation of the anti-thrombotic activity of a combination sirolimus and cilostazol drug eluting stent in an in vitro bovine blood loop model in accordance with the present invention.

FIG. 13 is a graphical representation of the in vivo release kinetics of sirolimus and cilostazol from the stent illustrated in FIG. 15.

FIG. 14 is a graphical representation of the in vitro release kinetics of sirolimus and cilostazol from the stent illustrated in FIG. 15.

FIG. 15 is a diagrammatic representation of a fourth exemplary embodiment of a stent coated with a combination of sirolimus and cilostazol in accordance with the present invention.

FIG. 16 is a graphical representation of the in vivo release kinetics of sirolimus and cilostazol from the stent illustrated in FIG. 7.

FIG. 17 is a graphical representation of the in vitro release kinetics of sirolimus and cilostazol from the stent illustrated in FIG. 7.

FIG. 18 is a graphical representation of the in vivo release kinetics of sirolimus and cilostazol from a dual drug stent in accordance with the present invention.

FIG. 19 is a graphical representation of the in vitro release kinetics of ATL-359 from a stent in accordance with the present invention.

### DETAILED DESCRIPTION OF THE PREFERRED EMBODIMENTS

While exemplary embodiments of the invention will be described with respect to treating or reducing myocardial injury following an acute myocardial infarction, it is important to note that the local delivery of drug/drug combinations may be utilized to treat a wide variety of conditions utilizing any number of medical devices, or to enhance the function and/or life of the device. For example, intraocular lenses, placed to restore vision after cataract surgery is often compromised by the formation of a secondary cataract. The latter is often a result of cellular overgrowth on the lens surface and can be potentially minimized by combining a drug or drugs with the device. Other medical devices which often fail due to tissue in-growth or accumulation of proteinaceous material in, on and around the device, such as shunts for hydrocephalus, dialysis grafts, colostomy bag attachment devices, ear drainage tubes, leads for pace makers and implantable defibrillators can also benefit from the device-drug combination approach. Devices which serve to improve the structure and function of tissue or organ may also show benefits when combined with the appropriate agent or agents. For example, improved osteointegration of orthopedic devices to enhance stabilization of the implanted device could potentially be achieved by combining it with agents such as bone-morphogenic protein. Similarly other surgical devices, sutures, staples, anastomosis devices, vertebral disks, bone pins, suture anchors, hemostatic barriers, clamps, screws, plates, clips, vascular implants, tissue adhesives and sealants, tissue scaffolds, various types of dressings, bone substitutes, intraluminal devices, and vascular supports could also provide enhanced patient benefit using this drug-device combination approach. Perivascular wraps may be particularly advantageous, alone or in combination with other medical devices. The perivascular wraps may supply additional drugs to a treatment site. Essentially, any type of medical device may be coated or loaded in some fashion with a drug or drug combination which enhances treatment over use of the singular use of the device or pharmaceutical agent.

In addition to various medical devices, the coatings on these devices may be used to deliver therapeutic and pharmaceutic agents including: anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes

(L-asparaginase which systemically metabolizes L-asparagine and deprives cells which do not have the capacity to synthesize their own asparagine); antiplatelet agents such as glycoprotein (GP) II_{b}/IIIₐ inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), azathioprine, mycophenolate mofetil); angiogenic agents: vascular endothelial growth factor (VEGF), fibroblast growth factor (FGF); angiotensin receptor blockers; nitric oxide donors; antisense oligionucleotides and combinations thereof; cell cycle inhibitors, mTOR inhibitors such as sirolimus, everolimus and other rapamycin analogs, and growth factor receptor signal transduction kinase inhibitors; retenoids; cyclin/CDK inhibitors; HMG co-enzyme reductase inhibitors (statins); and protease inhibitors.

A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen. However, self-expanding stents may be utilized without the need for a balloon.

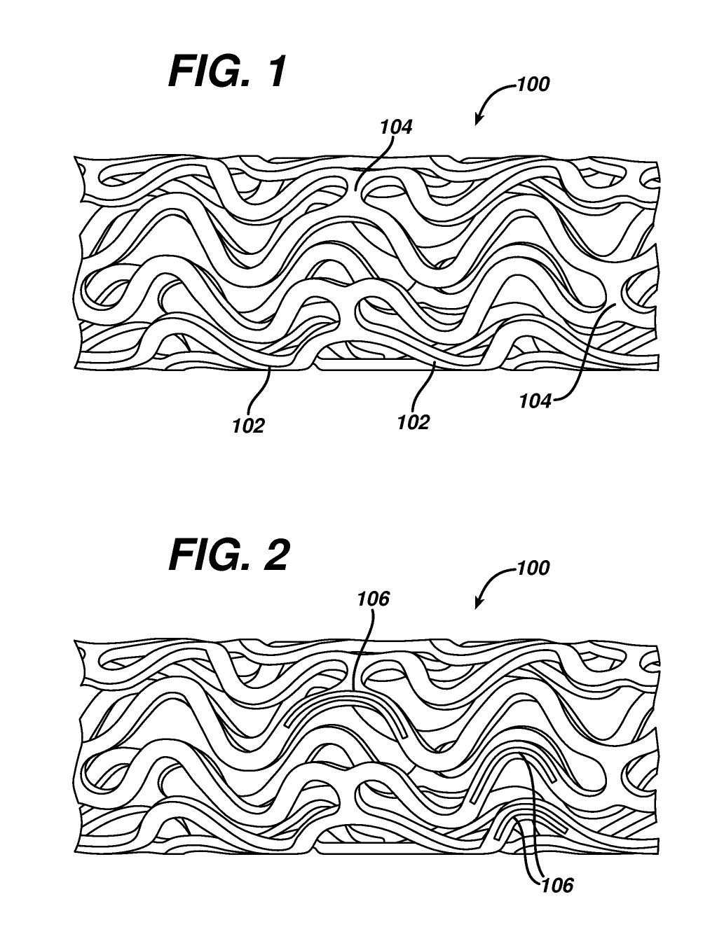

FIG. 1 illustrates an exemplary stent 100 which may be utilized in accordance with an exemplary embodiment of the present invention. The expandable cylindrical stent 100 comprises a fenestrated structure for placement in a blood vessel, duct or lumen to hold the vessel, duct or lumen open, more particularly for protecting a segment of artery from restenosis after angioplasty. The stent 100 may be expanded circumferentially and maintained in an expanded configuration that is circumferentially or radially rigid. The stent 100 is axially flexible and when flexed at a band, the stent 100 avoids any externally protruding component parts.

The stent 100 generally comprises first and second ends with an intermediate section therebetween. The stent 100 has a longitudinal axis and comprises a plurality of longitudinally disposed bands 102, wherein each band 102 defines a generally continuous wave along a line segment parallel to the longitudinal axis. A plurality of circumferentially arranged links 104 maintain the bands 102 in a substantially tubular structure. Essentially, each longitudinally disposed band 102 is connected at a plurality of periodic locations, by a short circumferentially arranged link 104 to an adjacent band 102. The wave associated with each of the bands 102 has approximately the same fundamental spatial frequency in the intermediate section, and the bands 102 are so disposed that the wave associated with them are generally aligned so as to be generally in phase with one another. As illustrated in the figure, each longitudinally arranged band 102 undulates through approximately two cycles before there is a link to an adjacent band 102.

The stent 100 may be fabricated utilizing any number of methods. For example, the stent 100 may be fabricated from a hollow or formed stainless steel tube that may be machined using lasers, electric discharge milling, chemical etching or other means. The stent 100 is inserted into the body and placed at the desired site in an unexpanded form. In one exemplary embodiment, expansion may be affected in a blood vessel by a balloon catheter, where the final diameter of the stent 100 is a function of the diameter of the balloon catheter used as well as the design (expansion ratio) of the stent.

It should be appreciated that a stent 100 in accordance with the present invention may be embodied in a shape-memory material, including, for example, an appropriate alloy of nickel and titanium or stainless steel. Structures formed from stainless steel may be made self-expanding by configuring the stainless steel in a predetermined manner, for example, by twisting it into a braided configuration. In this embodiment, after the stent 100 has been formed it may be compressed so as to occupy a space sufficiently small as to permit its insertion in a blood vessel or other tissue by insertion means, wherein the insertion means include a suitable catheter, or flexible rod. On emerging from the catheter, the stent 100 may be configured to expand into the desired configuration where the expansion is automatic or triggered by a change in pressure, temperature or electrical stimulation.

FIG. 2 illustrates an exemplary embodiment of the present invention utilizing the stent 100 illustrated in FIG. 1 with minor modifications. As illustrated, the stent 100 may be modified to comprise one or more reservoirs 106. Each of the reservoirs 106 may be opened or closed as desired. These reservoirs 106 may be specifically designed to hold the drug/drug combinations to be delivered. Regardless of the design of the stent 100, it is preferable to have the drug/drug combination dosage applied with enough specificity and a sufficient concentration to provide an effective dosage for the condition to be treated. In this regard, the reservoir size in the bands 102 is preferably sized to adequately apply the drug/drug combination dosage at the desired location and in the desired amount. However, it is important to note that the stent illustrated in FIG. 1 may also be utilized to deliver drug/drug combinations. For example, the surface of the stent may be coated directly with drug/drug combinations or as part of a polymeric matrix affixed to the surface of the stent. In other words, the stent surface coating is or acts as the drug delivery depot.

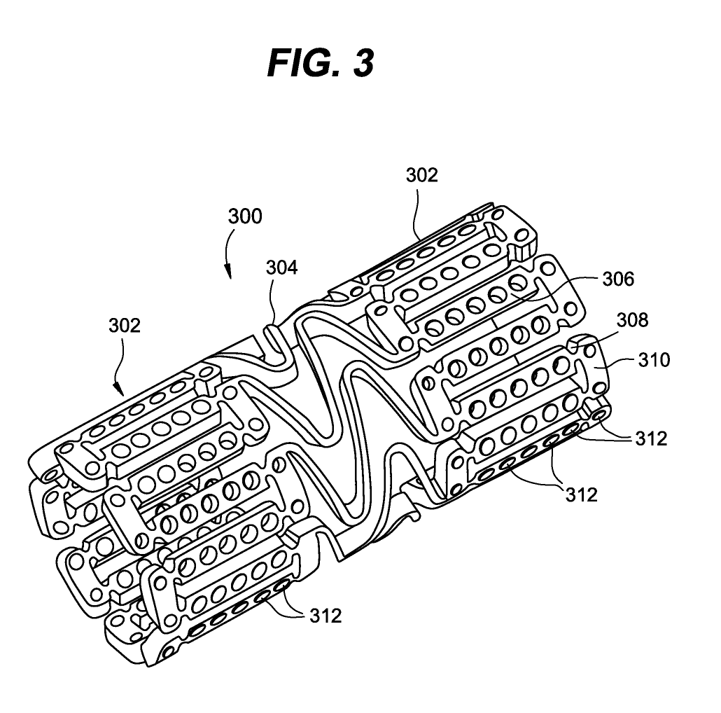

FIG. 3 illustrates an alternate exemplary expandable medical device having a plurality of through-holes containing a beneficial agent for delivery to tissue or into the bloodstream by the expandable medical device. The expandable medical device 300 illustrated in FIG. 3 is cut from a tube of material to form a cylindrical expandable device. The expandable medical device 300 includes a plurality of cylindrical sections 302 interconnected by a plurality of bridging elements 304. The bridging elements 304 allow the tissue supporting device to bend axially when passing through the torturous path of vasculature to a deployment site and allow the device to bend axially when necessary to match the curvature of a lumen to be supported. Each of the cylindrical sections 302 is formed by a network of elongated struts 306 which are interconnected by ductile hinges 308 and circumferential struts 310. During expansion of the medical device 300 the ductile hinges 308 deform while the struts 306 are not deformed.

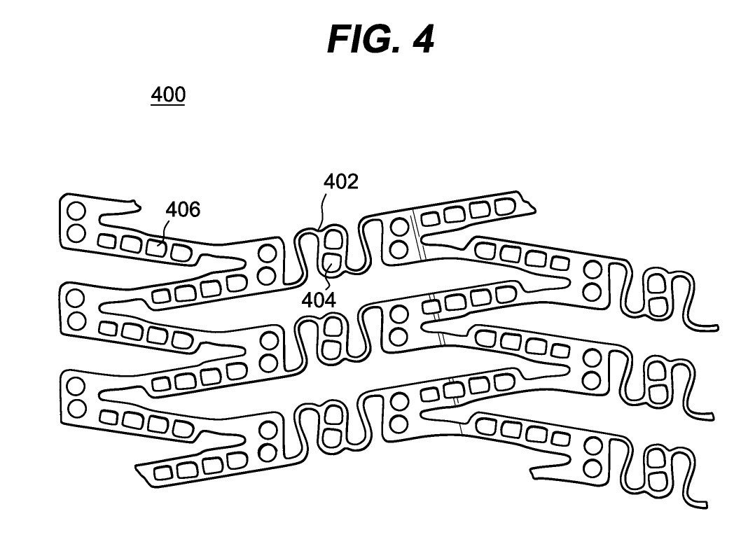

As illustrated in FIG. 3, the elongated struts 306 and circumferential struts 310 include openings 312, some or all of which contain a beneficial agent for delivery to the lumen in which the expandable medical device is implanted. In addition, other portions of the device 300, such as the bridging elements 304, may include openings, as illustrated in FIG. 4. In the device 400 illustrated in FIG. 4, the bridging elements 402 have a modified design from those illustrated in FIG. 3 to accommodate additional openings or reservoirs 404. Preferably, the openings or reservoirs 404 in the bridging elements 402 and the openings or reservoirs 406 in the remaining portions of the device 400 are provided in non-deforming portions of the device 400 so that the openings are non-deforming and the beneficial agent is delivered without risk of being fractured, expelled, or otherwise damaged during expansion of the device.

The exemplary embodiments of the stent of the present invention illustrated in FIG. 3 may be further refined by using Finite Element Analysis and other techniques to optimize the deployment of the beneficial agents within the openings 312. Basically, the shape and location of the openings 312, may be modified to maximize the volume of the voids while preserving the relatively high strength and rigidity of the struts with respect to the ductile hinges 308. Typically, the openings 312 are less than one hundred (100) percent filled for any application.

In accordance with exemplary embodiments of the present invention, single beneficial agents may be loaded into the reservoirs or holes in the stent or coated onto the surface thereof. In addition, multiple beneficial agents may be loaded into the reservoirs or holes in the stent or coated onto the surface thereof. The use of reservoirs or holes for drug or agent release as described above with respect to FIG. 3 makes using different beneficial agents easier as well as offering a number of advantages as set forth herein. Different beneficial agents comprising different drugs may be disposed in different openings in the stent. This allows the delivery of two or more beneficial agents from a single stent in any desired delivery pattern and with independent drug release rate profiles. Alternately, different beneficial agents comprising the same drug in different concentrations may be disposed in different openings. This allows the drug to be uniformly distributed to the tissue with a non-uniform device structure.

The two or more different beneficial agents provided in the devices described herein may comprise (1) different drugs; (2) different concentrations of the same drug; (3) the same drug with different release kinetics, i.e., different matrix erosion rates; or (4) different forms of the same drug. Examples of different beneficial agents formulated comprising the same drug with different release kinetics may use different carriers to achieve the elution profiles of different shapes. Some examples of different forms of the same drug include forms of a drug having varying hydrophilicity or lipophilicity.

In addition to the use of different beneficial agents in different openings to achieve different drug concentrations at different defined areas of tissue or in the bloodstream, the loading of different beneficial agents in different openings may be used to provide a more even spatial distribution of the beneficial agent delivered in instances where the expandable medical device has a non-uniform distribution of openings in the expanded configuration.

The use of different drugs in different openings in an interspersed or alternating manner allows the delivery of two different drugs which may not be deliverable if combined within the same polymer/drug matrix composition. For example, the drugs themselves may interact in an undesirable way. Alternatively, the two drugs may not be compatible with the same polymers for formation of the matrix or with the same solvents for delivery of the polymer/drug matrix into the openings.

Given that the openings in the stent of FIG. 3 are through holes, the construct of the loading of the openings with the one or more beneficial agents may be utilized to determine the direction of the release of the one or more beneficial agents, for example, predominantly to the luminal or abluminal side of the expandable medical device. In addition to the delivery of different beneficial agents to the mural or abluminal side of the expandable medical device for treatment of the vessel wall, beneficial agents may be delivered to the luminal side of the expandable medical device to prevent or reduce thrombosis or to directly and locally deliver agents into the bloodstream for the treatment of organs downstream of the implantation site as discussed in detail subsequently. Drugs which are delivered into the blood stream from the luminal side of the device may be located at a proximal end of the device, a distal end of the device or at desired specified regions of the device.

The methods for loading beneficial agents into the different openings in an expandable medical device may include known techniques such as dipping and coating and also known piezoelectric micro-jetting techniques. Micro-injection devices may be computer controlled to deliver precise amounts of one or more liquid formulated beneficial agents to precise locations on the expandable medical device in a known manner. For example, a dual agent jetting device or process may deliver two agents simultaneously or sequentially into the openings. When the beneficial agents are loaded into through openings in the expandable medical device, a luminal side of the through openings may be blocked during loading by a resilient mandrel allowing the beneficial agents to be delivered in liquid form, such as with a solvent. The beneficial agents may also be loaded by manual injection devices.

In accordance with the present invention, a stent with holes or reservoirs comprising a selective adenosine receptor agonist is described herein. However, it is important to note that the stent may be surface coated with a selective adenosine receptor agonist. For example, the selective adenosine receptor agonist may be applied directly to the surface of the device or mixed with a polymeric matrix and then affixed to the surface of the device. In addition, other medical devices such as angioplasty balloons may be surface coated with a selective adenosine receptor agonist and utilized to locally deliver the agent to the desired treatment location with or without a stent or other intraluminal scaffold. When utilizing a stent with reservoirs, it may be possible to achieve various release rates and various agent concentrations or doses. For example, the drug may be selectively released in different phases and/or at different dosages depending on time. This may be achieved by filling the different reservoirs with material that can alter the elution rate of the drug, by utilizing different concentrations of the same drug and/or different forms of the same drug.

Adenosine receptors comprise four member subfamilies of G protein coupled receptors designated as A₁, A_{2A}, A_{2B} and A₃. Each of the four subtypes have selective agonists of which over a dozen are in, are undergoing or have been in clinical trials for the treatment of various conditions. In the exemplary embodiments described herein, the selective adenosine receptor agonist is an adenosine A_{2A} receptor agonist. However, it is important to note that the other selective adenosine receptor agonists may be utilized.

A stent or other implantable medical device for the local delivery of an adenosine A_{2A} receptor agonist may be utilized to reduce myocardial injury following an acute myocardial infarction. As soon as possible following an acute myocardial infarction a stent or other suitable device comprising and capable of delivering an adenosine A_{2A} receptor agonist is positioned in the blood vessel with the occlusion responsible for causing the infarct. Once in position , the stent or other intraluminal device is deployed to remove the occlusion and reestablish blood flow to the specific area, region or tissue volume of the heart. Over a given period of time described in detail subsequently, the adenosine A_{2A} receptor agonist elutes from the stent or other device into the downstream coronary blood flow into the hypoxic cardiac tissue for a time sufficient to reduce the level of myocardial injury.

The early and sustained release of the adenosine A_{2A} receptor agonist may reduce myocardial injury be reducing the size or amount of infarcted myocardial tissue, reducing the level of myocellular death, reduce the extent of reperfusion injury, preserve more function in the myocapillary bed and or mitigate the so-called “no-reflow” condition which should in turn improve cardiac output, ejection fraction and cardiac wall motion post infarct. The delivery of the adenosine A_{2A} receptor agonist from the stent or other device to the hypoxic tissue will begin immediately after the occluded vessel has been made patent by deployment of the device (reperfusion), or more specifically, the delivery of the agent from the device will not begin until blood flow is reestablished to the tissue of the treatment site as the blood caries the agent. In the case of a drug eluting stent or reservoir eluting stent, delivery of the adenosine A_{2A} receptor agonist will begin immediately upon expansion of the stent and removal of the balloon which will allow the agonist to elute. If a self expanding stent is utilized, adenosine A_{2A} receptor agonist delivery will begin upon deployment of the stent and contact with the blood.

The local delivery of the adenosine A_{2A} receptor agonist to the tissue at risk may be continued from the time the artery is recanalized for a period of one (1) to seventy-two (72) hours. Preferably, the adenosine A_{2A} receptor agonist is delivered for a period of between four (4) and twenty-four (24) hours post infarct. The amount of adenosine A_{2A} receptor agonist delivered to the hypoxic tissue over the given time period is up to about 2 milligrams or 2000 micrograms. The adenosine A_{2A} receptor agonist utilized in the present invention is preferably a high potency adenosine A_{2A} receptor agonist with an activity greater than adenosine itself, such as ATL-359 available from PGxHealth LLC. Other adenosine A_{2A} receptor agonists include ATL-1222 and/or ATL-146e both of which are also available from PGxHealth LLC. A detailed description of the elution profile as well as the therapeutic agent complex to be loaded into the stent is set forth subsequently.

Adenosine has a number of properties, including coronary vasodilator, anti-inflammatory, mediator of ischemic preconditioning and the reduction of no-reflow and infarct size. Adenosine receptor agonists, as set forth herein, have been identified that are over one hundred times more potent than adenosine as coronary vasodilators with the potential to improve coronary perfusion and reduce infarct size. However, it is important to note that the adenosine receptor agonists set forth therein may be locally delivered to treat ischemic tissue elsewhere in the body, including the brain.

In a typical drug eluting device, the drug is mixed with a number of constituents such as polymers. Any number of biocompatible polymers may be utilized. The polymer that serves to hold the drug in the reservoir cavity and modulate the elution rate of the drug is preferably a bioresorbable polymer. Examples of bioresorbable polymers include, but are not limited to, poly-α-hydroxy acid esters such as, polylactic acid (PLLA or DL-PLA), polyglycolic acid, polylactic-co-glycolic acid (PLGA), polylactic acid-co-caprolactone, poly (block-ethylene oxide-block-lactide-co-glycolide) polymers (PEO-block-PLGA and PEO-block-PLGA-block-PEO); polyethylene glycol (PEG) and polyethylene oxide (PEO), poly (block-ethylene oxide-block-propylene oxide-block-ethylene oxide), Poloxamers; polyvinyl pyrrolidone (PVP); polyorthoesters (POE); polysaccharides and polysaccharide derivatives such as polyhyaluronic acid, heparin, poly (glucose), poly(alginic acid), chitin, chitosan, chitosan derivatives, cellulose, methyl cellulose, hydroxyethylcellulose, hydroxypropylcellulose, carboxymethylcellulose; polypeptides, and proteins such as polylysine, polyglutamic acid, albumin; polyanhydrides; polyhydroxy alkonoates such as polyhydroxy valerate, polyhydroxy butyrate, and the like.

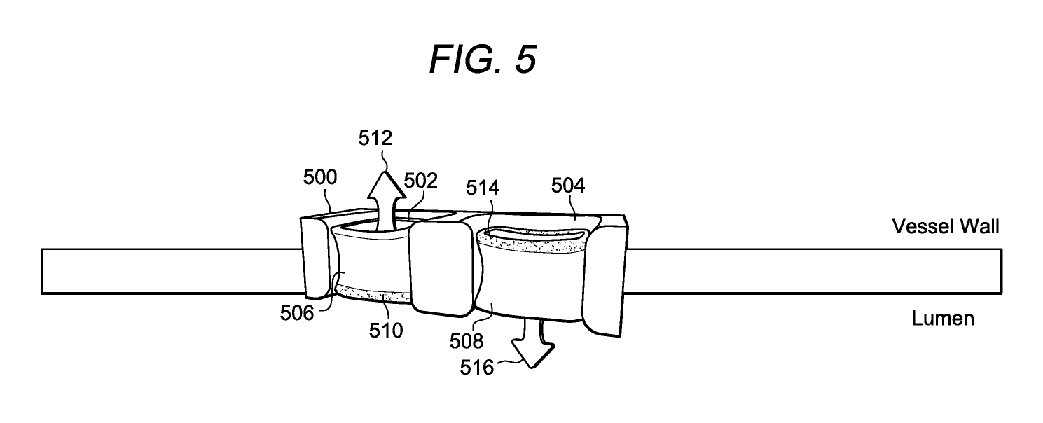

FIG. 5 is a diagrammatic, side view representation of a portion of a drug eluting stent in accordance with the present invention. Although the pattern for therapeutic agent or drug delivery may be tailored for a number of different situations or treatment scenarios as described above, for ease of explanation adjacent reservoirs are described as comprising two different drugs. The drug eluting stent 500 is illustrated comprising two reservoirs 502 and 504, one being filled with a first composition 506 and the other being filled with a second composition 508. A barrier layer 510 may be positioned on the luminal side of the stent 500 to cause the first composition 506 to elute predominantly towards the vessel wall and into the tissue comprising the vessel wall as illustrated by arrow 512. A barrier layer 514 may be positioned on the abluminal side of the stent 500 to cause the second composition 508 to elute predominantly towards the lumen of the vessel and into the bloodstream as indicated by arrow 516. As illustrated, the use of barrier layers may be easily utilized to control the direction of elution. In the present invention, for the reasons set forth herein, it is preferable that the adenosine A_{2A} receptor agonist elute into the bloodstream for downstream treatment of an organ such as the heart and preferably the region of the heart fed or perfused by the formerly occluded vessel.

FIG. 5 illustrates the compositions and the barrier layers as distinct regions within the openings; however, it should be understood that these regions are not totally distinct regions in that they are formed by a blending of the different regions and the materials that comprise them. Thus, although the barrier layers are primarily polymer without drug, depending on the manufacturing processes employed, some small amount of drug of the subsequent region can be incorporation into the barrier region.

As described above, the reservoirs of the stent may be filled or loaded in any number of ways. In the exemplary embodiment, the compositions are filled or loaded into the reservoir wells or reservoirs in two separate and sequential series of steps, including firstly depositing a fluid filling solution composition into the reservoirs and secondly evaporating the majority, if not substantially all, of the filling solution solvent. Having no solvent is the ideal situation; however, current processes and materials do not result in an absolutely no residual solvent mix. The compositions in accordance with the present invention as described herein are the solid materials that remain in the reservoirs after removal of substantially all and preferably all of the solvent from the filling solution composition.

The fluid compositions used to form the solid composition comprising adenosine A_{2A} receptor agonists include a bioresorbable or bioabsorbable polymer, preferably a poly(lactide-co-glycolide), PLGA, polymer, a suitable solvent such as dimethyl sulfoxide, DMSO, or N-methyl pyrrolidone, NMP, an adenosine A_{2A} receptor agonist such as ATL-359 and optionally a stabilizer or anti-oxidant such as BHT. Alternatives for DMSO and NMP include dimethyl acetamide (DMAc) or dimethyl formamide (DMF). DMSO is preferred because ATL-359 is more stable in the presence of DMSO and DMSO is a more bio-friendly solvent.

Each sequential fluid composition that is deposited may comprise the same ingredients, or sequential filling solutions may be prepared from filling solutions comprising different ingredients. Preferably, the first series of filling solution deposits comprise polymer, therapeutic agent and solvent, which are dried after each filling step. This part of the process results in the formation or construct of the main therapeutic agent structure. The second series of filling solution deposits comprise only polymer and solvent, which are dried after each filling step. This manufacturing sequence will create a reservoir composition in which there is a higher concentration of ATL-359 in the area of the luminal face of the reservoir and a relatively lower concentration of ATL-359 in the area of the abluminal face of each reservoir. Such a configuration, as described in detail above, creates a longer path or higher resistance to elution of the drug to the abluminal face as compared to the luminal face and as such should result in substantially all of the ATL-359 being delivered to the luminal side of the stent and into the arterial bloodflow. In other words, the reservoirs that deliver ATL-359 in a predominantly luminal direction will have a design where the volume of the reservoir on and near the abluminal surface of the stent will be comprised predominantly of polymer and a minor amount of ATL-359, while the volume of the same reservoir at or near the luminal surface will be comprised predominantly of ATL-359 with a minor proportion of polymer.

The adenosine A_{2A} receptor agonist composition within a reservoir will preferably comprise adenosine A_{2A} receptor agonist, a bioresorbable polymer, a solvent and optionally a stabilizing agent, and be in certain proportions to one another. Preferably, the total dose or amount of ATL-359 available from the drug eluting stent is between 10 and 2000 micrograms and more preferably between 30 and 450 micrograms (for a 3.5×17 mm stent) which from a 3.5×17 mm stent would be between 0.2 and 2.75 micrograms per square millimeter of arterial tissue area, where the area of arterial tissue is defined as the area of the surface of a theoretical cylinder whose diameter and length are the diameter and length of the expanded stent as deployed in the artery. The total delivered dose of ATL-359 or other adenosine A_{2A} receptor agonist will scale with the stent diameter and length.

As set forth above, the bioresorbable polymer utilized in the composition comprises PLGA. More preferably, the composition comprises a PLGA polymer where the molar ratio of lactide to glycolide residues (L:G) in the polymer chain is from about 85:15 to about 65:35. Even more preferably, the composition comprises a PLGA polymer where the molar ratio of lactide to glycolide residues (L:G) in the polymer chain is from about 80:20 to about 70:30. The PLGA should preferably have an intrinsic viscosity in the range from about 0.1 to about 0.9 dL/g. In the exemplary embodiment, the composition comprises a PLGA polymer where the molar ratio of lactide to glycolide (L:G) in the polymer chain is 75/25 in both the drug composition and the barrier layer with an intrinsic viscosity of 0.68 in the drug composition and 0.21 in the barrier layer. The weight ratio of ATL-359 to PLGA, designated as the D/P ratio, is preferably in the range from 95/5 with a four (4) percent volume cap and a dose of about 523 micrograms on a 3.5×17 mm stent for an overall D/P of 85.7/14.3 to about 60/40 with an eighteen (18) percent cap and dose of 275 micrograms for an overall D/P of 47.5/52.5. These values are scalable with dose and stent size. All ratios are weight percentages.

In order to make the above-described constituents a solution for filling purposes, a suitable solvent is required. Dimethyl sulfoxide, DMSO is the preferred solvent and is preferably utilized in an amount of ATL-359 in the range from about 1 percent to about 30 percent by weight relative to the total weight of DMSO filling solution. Even more preferably ATL-359 is utilized in an amount in the range from about 10 percent to about 25 percent by weight relative to the total weight of DMSO filling solution. Even yet more preferably ATL-359 is utilized in an amount in the range from about 15 percent to about 21 percent by weight relative to the total weight of DMSO filling solution.

It is important to note that the drug loading or doses for each drug may be expressed in any number of ways, including those set forth above. In a preferred exemplary embodiment, the dose ranges may be expressed as nested absolute ranges of drug weight based on a standard 3.5 mm×17 mm stent size. In this way, the dose ranges would scale with stent size and reservoir count. For example, in a 3.5 mm×17 mm stent size the number of holes or reservoirs is 585. In other exemplary embodiments, the number of reservoirs for a given size stent may include 211 reservoirs for a 2.5 mm×8 mm stent, 238 for a 3.0 mm×8 mm stent, 290 reservoirs for a 3.5 mm×8 mm stent, 311 reservoirs for a 2.5 mm×12 mm stent, 347 for a 3.0 mm×12 mm stent, 417 reservoirs for a 3.5 mm×12 mm stent, 431 reservoirs for a 2.5 mm×17 mm stent, 501 for a 3.0 mm×17 mm stent, 551 reservoirs for a 2.5 mm×22 mm stent, 633 for a 3.0 mm×22 mm stent, 753 reservoirs for a 3.5 mm×22 mm stent, 711 reservoirs for a 2.5 mm×28 mm stent, 809 for a 3.0 mm×28 mm stent, 949 reservoirs for a 3.5 mm×28 mm stent, 831 reservoirs for a 2.5 mm×33 mm stent, 963 for a 3.0 mm×33mm stent and 1117 reservoirs for a 3.5 mm×33 mm stent.

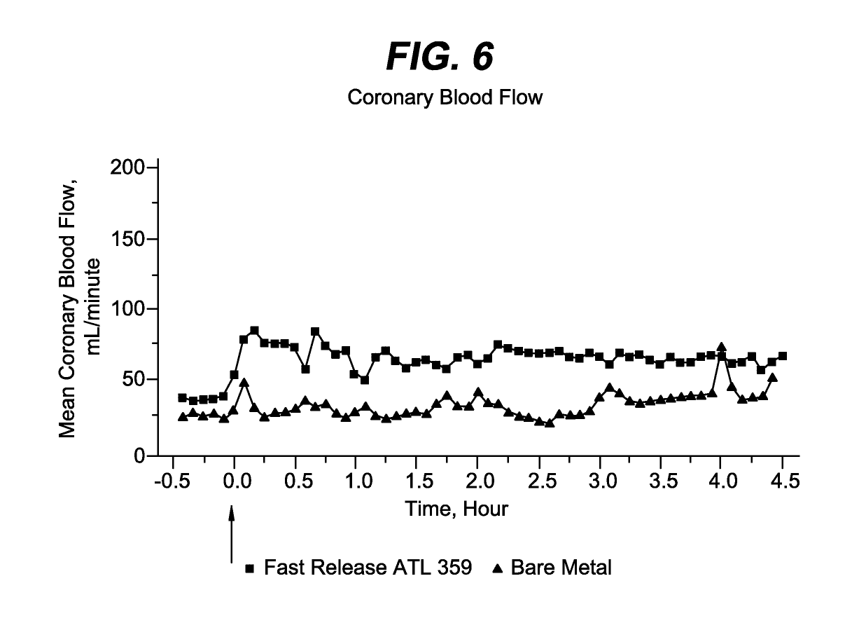

FIG. 6 graphically illustrates the improved blood flow through a stent releasing ATL-359 into the blood stream as compared to the blood flow through a bare metal stent. Curve 602 is the measured blood flow through bare metal stents and curve 604 is the measured blood flow through stents eluting ATL-359. As can be readily seen from a comparison of the two curves, ATL-359 released from a stent results in a significantly higher blood flow.

The data from which the curves 602 and 604 were generated were the result of an experimental protocol involving anesthetized domestic pigs. In this experiment, a single stent (3.0×17 mm) was implanted into the mid-LAD coronary artery under fluoroscopy via femoral access in a pig anesthetized using isoflurane gas. A total of nine pigs were utilized. In six of the pigs the stents contained ATL-359 as described above and in the remaining three pigs the same stents were utilized, but with no ATL-359. Once implanted with the stents, continuous hemodynamic recording was performed for four hours with the results illustrated in FIG. 6.

Other selective agonists include Selodenoson (DT10009), Tecadenoson (CVT-510), CVT-2759, Binodenoson (MRE0470), Regadenoson (CVT-3146), MRE0094, BAY-60-6583), CF101 (IB-MECA), CF102 (CI-IB-MECA), CF502 (MRS3558) and AMP-579.

The drug eluting stent of the present invention may be utilized to treat a number of disease states as set forth above, including restenosis, thrombosis, acute myocardial infarction, reperfusion injury, capillary no-reflow conditions, and ischemic related conditions. In addition to the use of adenosine A_{2A} receptor agonist, other drugs may be added to the device. For example, anti-thrombotic agents such as heparin, cilostazol or tirofiban may be added. The additional drugs may be included as coatings or in reservoirs. What is important to note is that any number of drugs and reservoir combinations as well as coatings may be utilized to tailor the device to a particular disease state. For example, sirolimus which is a known effective inhibitor of smooth muscle cell growth may be utilized in combination with a selective adenosine receptor agonist to provide an effective means for treating restenosis. Specifically, in the stent illustrated in FIG. 5, the rapamycin may be delivered into the vessel wall while the adenosine receptor agonist may be delivered into the blood stream. With this same device, heparin or a similar drug may be affixed to the non-reservoir surface and thus a single device may be utilized to treat restenosis, hypoxic tissue and thrombosis.

In accordance with another exemplary embodiment, additional therapeutic agents may be utilized in combination to treat acute myocardial infarction, ischemia and/or reperfusion injury as well as other injury-related conditions such as restenosis. Through the introduction of the one or more agents described herein via delivery from a stent or other suitable device, the combination of agents may increase intracellular levels of cyclic adenosine monophosphate (cAMP) in local target cells (smooth muscle, endothelial, inflammatory and cardiac muscle cells). Elevating cAMP levels is associated with smooth muscle vasodilatation and inhibition of proliferation, decreased platelet activation, decreased inflammation, and positive inotropy. Agents that increase cAMP include phosphodiesterase inhibitors (PDEi), which inhibit the enzymatic degradation of cAMP to 5′-AMP, and adenosine A_{2A} receptor agonists which are linked to activation of adenyl cyclase, responsible for converting ATP to cAMP. PDEi can be utilized with the adenosine A_{2A} receptor agonists described above and with a rapamycin to achieve benefits that may exceed the use of the individual agents alone as is explained in detail subsequently.

Preferably, the initiation of delivery of the PDEi from the stent or other device will be immediately or very shortly after deployment of the drug eluting or reservoir eluting stent and restoration of blood flow to the coronary vasculature affected in the acute myocardial infarct event. The delivery of a

PDEi from a stent or other medical device will preferably result in improved blood flow distribution to the ischemic tissue, a reduction in the amount of cardiac tissue that is infarcted or becomes necrotic, the degree of damage in so-called reperfusion injury that occurs when blood flow is reestablished to the affected tissue by the opening of the occluded coronary artery with the stent or other device, and the longer term ischemia-related effects on the tissue. Once again, it is important to note that the device and therapeutic agents described herein may be utilized in the treatment of other organs.

Phosphodiesterase Type III inhibitors, or PDE-III active compounds are a subset of PDEi that are expected to provide the greatest benefit in coronary vessels, including smaller arterioles and capillaries and myocardial tissue. These agents include, cilostazol, milrinone, inamrinone, cilostamide, saterinone, motapizone, lixazinone, enoximone (fenoximone), imazodan, pimobendan. Non-specific active PDE compounds that are much less preferred, but may have utility, include theophylline and aminophylline. PDE-V active compounds (cyclic GMP specific phosphodiesterase inhibitors) that may also have utility, include sildenafil and tadalafil. Preferably, the PDEi is a type III PDEi and more preferably the PDEi is cilostazol which has other benefits as detailed below.

In the exemplary embodiments set forth below, the use of a rapamycin and cilostazol is from a drug coated stent as opposed to a reservoir eluting stent. In addition, different polymers are utilized for the drug matrix. However, it is important to note that the use of these two drugs in combination with an adenosine A_{2A} receptor agonist, as described above, will work equally well from a reservoir eluting stent as described and illustrated herein. In other words, the rapamycin, cilostazol and the selective adenosine A_{2A} receptor agonist may be loaded into the reservoirs in any combination desired to achieve the desired therapeutic effects as is explained in detail subsequently. The exemplary embodiments set forth below illustrate the effectiveness of a combination of a rapamycin and a PDEi in the treatment of restenosis and thrombosis.

Rapamycin is a macrocyclic triene antibiotic produced by Streptomyces hygroscopicus as disclosed in U.S. Pat. No. 3,929,992. It has been found that rapamycin among other things inhibits the proliferation of vascular smooth muscle cells in vivo. Accordingly, rapamycin may be utilized in treating intimal smooth muscle cell hyperplasia, restenosis, and vascular occlusion in a mammal, particularly following either biologically or mechanically mediated vascular injury, or under conditions that would predispose a mammal to suffering such a vascular injury. Rapamycin functions to inhibit smooth muscle cell proliferation and does not interfere with the re-endothelialization of the vessel walls.

Rapamycin reduces vascular hyperplasia by antagonizing smooth muscle proliferation in response to mitogenic signals that are released during an angioplasty induced injury. Inhibition of growth factor and cytokine mediated smooth muscle proliferation at the late G1 phase of the cell cycle is believed to be the dominant mechanism of action of rapamycin. However, rapamycin is also known to prevent T-cell proliferation and differentiation when administered systemically. This is the basis for its immunosuppressive activity and its ability to prevent graft rejection.

As used herein, rapamycin includes rapamycin and all analogs, derivatives and conjugates that bind to FKBP12, and other immunophilins and possesses the same pharmacologic properties as rapamycin including inhibition of TOR.

Although the anti-proliferative effects of rapamycin may be achieved through systemic use, superior results may be achieved through the local delivery of the compound. Essentially, rapamycin works in the tissues, which are in proximity to the compound, and has diminished effect as the distance from the delivery device increases. In order to take advantage of this effect, one would want the rapamycin in direct contact with the lumen walls.

As disclosed above, rapamycin may be utilized in combination with cilostazol. Cilostazol {6[4-(1-cyclohexyl-1H-tetrazol-5-yl)-butoxy]-3,4-dihydro-2-(1H)-quinolinone} is an inhibitor of type III (cyclic AMP specific) phosphodiesterase and has anti-platelet and vasodilator properties. Cilostazol was originally developed as a selective inhibitor of cyclic nucleotide phosphodiesterase 3. Phosphodiesterase 3 inhibition in platelets and vascular smooth muscle cells was expected to provide an anti-platelet effect and vasodilation; however, preclinical studies have demonstrated that cilostazol also possesses the ability to inhibit adenosine uptake by various cells, a property that distinguishes cilostazol from other phosphodiesterase 3 inhibitors, such as milrinone. Accordingly, cilostazol has been shown to have unique antithrombotic, vasodilatory and cardioprotective properties based upon a number of novel mechanisms of action.

Studies have also shown the efficacy of cilostazol in reducing restenosis after the implantation of a stent. See, for example, Matsutani M., Ueda H. et al.: “Effect of cilostazol in preventing restenosis after percutaneous transluminal coronary angioplasty, Am. J. Cardiol 1997, 79:1097-1099, Kunishima T., Musha H., Eto F., et al.: A randomized trial of aspirin versus cilostazol therapy after successful coronary stent implantation, Clin Thor 1997, 19:1058-1066, and Tsuchikane E. Fukuhara A., Kobayashi T., et al.: Impact of cilostazol on restenosis after percutaneous coronary balloon angioplasty, Circulation 1999, 100:21-26.

In accordance with the present invention, cilostazol may be configured for sustained release from a medical device or medical device coating to help reduce platelet deposition and thrombosis formation on the surface of the medical device. As described herein, such medical devices include any short and long term implant in constant contact with blood such as cardiovascular, peripheral and intracranial stents. Optionally, cilostazol may be incorporated in an appropriate polymeric coating or matrix in combination with a rapamycin or other potent anti-restenotic agents.

The incorporation and subsequent sustained release of cilostazol from a medical device or a medical device coating will preferably reduce platelet deposition and thrombosis formation on the surface of the medical device. There is, as described above, pre-clinical and clinical evidence that indicates that cilostazol also has anti-restenotic effects partly due to its antiproliferative action.

Accordingly, cilostazol is efficacious on at least two aspects of blood contacting devices such as drug eluting stents. Therefore, a combination of cilostazol with another potent anti-restenotic agent including a rapamycin, such as sirolimus, its analogs, derivatives, congeners and conjugates or paclitaxel, its analogs, derivatives, congeners and conjugates may be utilized for the local treatment of cardiovascular diseases and reducing platelet deposition and thrombosis formation on the surface of the medical device. Although described with respect to stents, it is important to note that the drug combinations described with respect to this exemplary embodiment may be utilized in connection with any number of medical devices, some of which are described herein.

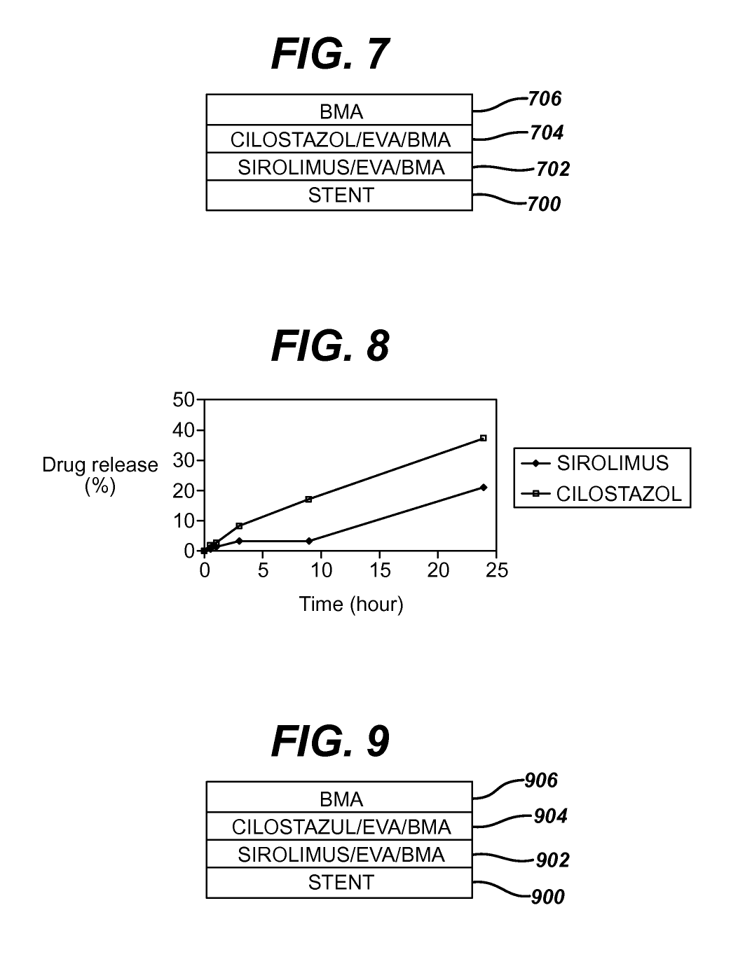

FIG. 7 illustrates a first exemplary configuration of a combination of cilostazol and a rapamycin on a stent. In this exemplary embodiment, the stent is a Bx Velocity® stent available from Cordis Corporation. In this particular configuration, the stent 700 is coated with three layers. The first layer or inner layer 702 comprises one hundred eighty (180 μg) micrograms of sirolimus which is equivalent to forty-five (45) percent by weight of the total weight of the inner layer 702 and a copolymer matrix of, polyethelene-co-vinylacetate and polybutylmethacrylate, EVA/BMA which is equivalent to fifty-five (55) percent by weight of the total weight of the inner layer 702. The second layer or outer layer 704 comprises one hundred (100 μg) micrograms of cilostazol which is equivalent to forty-five (45) percent by weight of the total weight of the outer layer 704 and a copolymer matrix of EVA/BMA which is equivalent to fifty-five (55) percent by weight of the total weight of the outer layer 704. The third layer or diffusion overcoat 706 comprises two hundred (200 μg) micrograms of BMA. The range of content recovery was eighty-five (85) percent of nominal drug content for the sirolimus and ninety-eight (98) percent of nominal drug content for cilostazol. The in vitro release kinetics for both cilostazol and sirolimus are illustrated in FIG. 8 and are described in more detail subsequently.

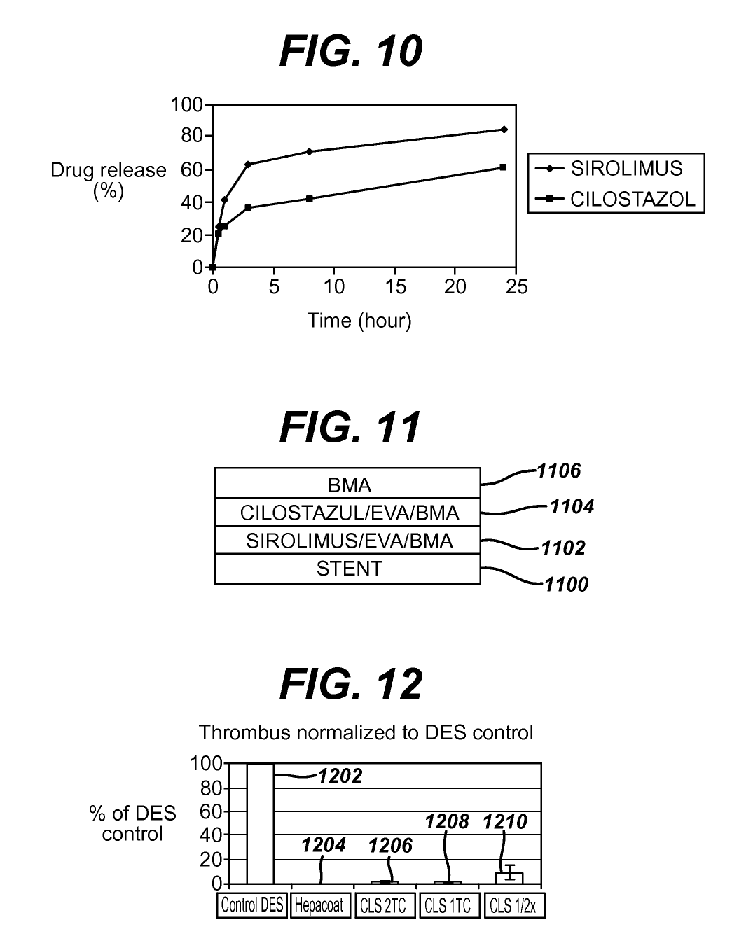

FIG. 9 illustrates a second exemplary configuration of a combination of cilostazol and a rapamycin on a stent. As described above, the stent is a Bx Velocity® stent available from Cordis Corporation. In this exemplary embodiment, the stent 900 is coated with three layers. The first layer or inner layer 902 comprises one hundred eighty (180 μg) micrograms of sirolimus which is equivalent to forty-five (45) percent by weight of the total weight of the inner layer 902 and a copolymer matrix of EVA/BMA which is equivalent to fifty-five (55) percent by weight of the total weight of the inner layer 902. The second layer or outer layer 904 comprises one hundred (100 μg) micrograms of cilostazol which is equivalent to forty-five (45) percent by weight of the total weight of the outer layer 904 and a copolymer matrix of EVA/BMA which is equivalent to fifty-five (55) percent by weight of the outer layer 904. The third layer or diffusion overcoat 906 comprises one hundred (100 =g) micrograms of BMA. Once again, the range of content recovery was eighty-five (85) percent of nominal drug content for the sirolimus and ninety-eight (98) percent of nominal drug content in cilostazol. The in-vitro release kinetics for both cilostazol and sirolimus are illustrated in FIG. 10 and are described in more detail subsequently.

As may be readily seen from a comparison of FIGS. 8 and 10, the drug release rate of both sirolimus and cilostazol was comparatively slower from the configuration comprising the thicker diffusion overcoat of BMA, i.e. two hundred micrograms rather than one hundred micrograms. Accordingly, additional control over the drug elution rates for both drugs may be achieved through the selective use of diffusion overcoats as described more fully herein. The selective use of diffusion overcoats includes thickness as well as other features, including chemical incompatibility.

FIG. 11 illustrates a third exemplary configuration of a combination of cilostazol and a rapamycin on a stent. This configuration is identical in structure to that of the configuration of FIG. 7, but with the amount of cilostazol reduced to fifty (50 μg) micrograms. As with the previous exemplary embodiment, there is a stent 1100 and three additional layers 1102, 1104 and 1106. The percentage by weight, however, remains the same.

The anti-thrombotic efficacy of the above-described three configurations is illustrated in FIG. 12. FIG. 12 illustrates the anti-thrombotic properties of the sirolimus/cilostazol combination coatings described above in an in vitro bovine blood loop model. In the in vitro bovine blood loop model, fresh bovine blood is heparinized to adjust for acute clotting time (ACT) of about two hundred (200) seconds. The platelet content in the blood is labeled through the use of Indium 111. In the study, a stent is deployed in a silicone tube, which is part of a closed loop system for blood circulation. The heparinzed blood is circulated through the closed loop system by means of a circulating pump. Activated platelets, fibrin and thrombus builds up on a stent surface over time and reduces the flow rate of blood through the stented loop. The flow is stopped when the flow rate is reduced to fifty (50) percent of the starting value or at ninety (90) minutes if none of the tested stent reduces the flow by fifty (50) percent. The total radioactivity (In 111) on the stent surface is counted by a beta counter and normalized with the control unit, set as one hundred (100) percent in the chart. A smaller number indicates that the surface is less thrombogenic. All three sirolimus/cilostazol dual drug coating groups reduced platelet deposition and thrombus formation on the stent surface by more than ninety (90) percent compared to the control drug eluting stent without the additional cilostazol compound. Bar 1202 represents the control drug eluting stent which has been normalized to one hundred (100) percent. The control drug eluting stent is the Cypher® sirolimus eluting coronary stent available from Cordis Corporation. Bar 1204 is a stent coated with heparin and is available from Cordis Corporation under the HEPACOAT® on the Bx Velocity® coronary stent trademark. Bar 1206 is a stent configured as set forth with respect to the architecture illustrated in FIG. 7. Bar 1208 is a stent configured as set forth with respect to the architecture illustrated in FIG. 9. Bar 1210 is a stent configured as set forth with respect to the architecture illustrated in FIG. 11. As may be readily seen from FIG. 12, cilostazol significantly reduces thrombus formation.

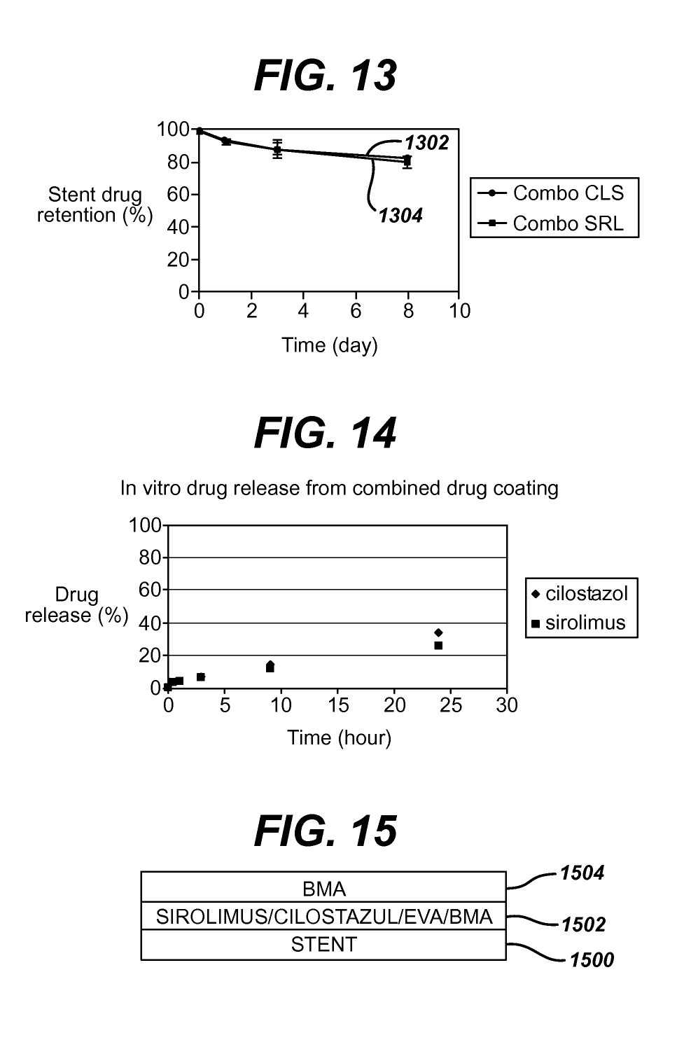

Another critical parameter for the performance of the thrombus resistance of a device coated with cilostazol is the duration of the drug release from the coating. This is of particular significance in the two weeks after device implantation. In the porcine drug elution PK studies of the dual drug eluting coating, both cilostazol and sirolimus were slowly released from the coating, resulting in a sustained drug release profile. The purpose of the porcine PK study is to assess the local pharmacokinetics of a drug eluting stent at a given implantation time. Normally three stents are implanted in three different coronary arteries in a pig for a given time point and then retrieved for total drug recovery analysis. The stents are retrieved at predetermined time points; namely, 1, 3 and 8 days. The stents are extracted and the total amount of drug remaining on the stents is determined by analysis utilizing HPLC (high performance liquid chromatography) for total drug amount. The difference between the original amount of drug on the stent and the amount of drug retrieved at a given time represents the amount of drug released in that period. The continuous release of drug into surrounding arterial tissue is what prevents the neointimal growth and restenosis in the coronary artery. A normal plot represents the percentage of total drug released (%, y-axis) vs. time of implantation (day, x-axis). As illustrated in FIG. 13, approximately eighty percent (80%) of the two drugs remained in the drug coating after eight (8) days of implantation. In addition, both drugs were released at a similar rate, despite the relatively large difference between their respective log P values and water solubility. Curve 1302 represents cilostazol and curve 1304 represents sirolimus. Their respective in vitro release profiles are illustrated in FIG. 14. Similar to the in vivo release profile, both sirolimus, represented by squares, and cilostazol, represented by diamonds, were released rather slowly, with only about thirty-five (35) percent release from both drugs. FIGS. 13 and 14 represent the in vivo and in vitro release rates from a stent coated in accordance with the configuration of FIG. 15 respectively, wherein the sirolimus and cilostazol are in one single layer, rather than in two separate layers. In this exemplary configuration, the stent 1500 is coated with two layers. The first layer 1502 comprises a combination of sirolimus, cilostazol and a copolymer matrix of EVA/BMA. The second layer or diffusion overcoat 1504 comprises only BMA. More specifically, in this embodiment, the first layer 1502 comprises a combination of sirolimus and cilastazol that is forty-five (45) percent by weight of the total weight of the first layer 1502 and an EVA/BMA copolymer matrix that is fifty-five (55) percent by weight of the total weight of the first layer 1502. The diffusion overcoat comprises one hundred (100 μg) micrograms of BMA.

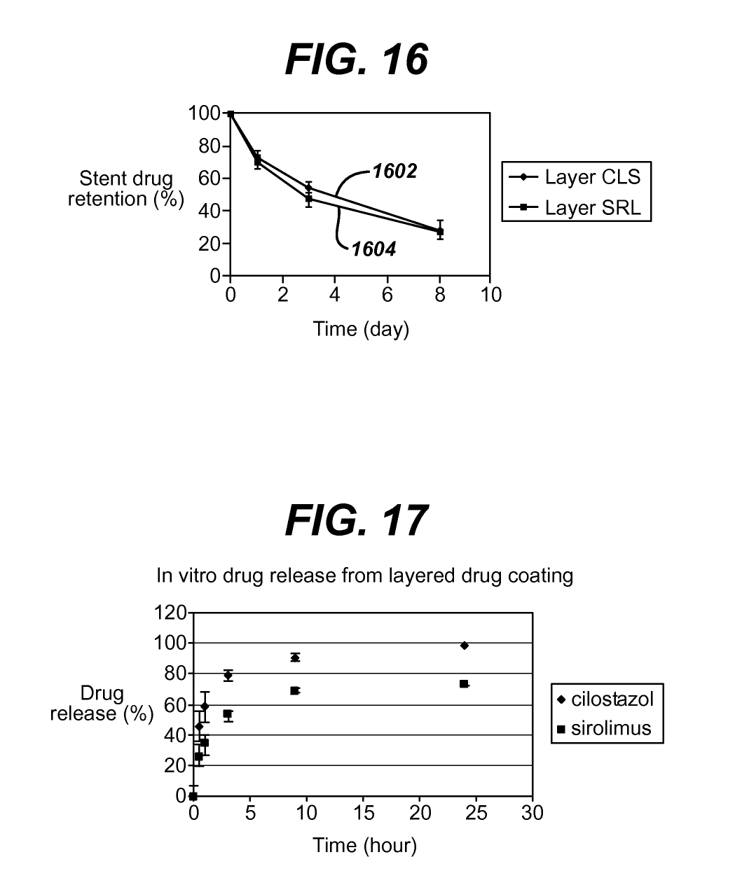

FIGS. 16 and 17 represent the in vivo and in vitro release rate from a stent coated in accordance with the configuration in FIG. 7, respectively. The layered dual drug eluting coating had a relatively faster release rate in the same procine PK model compared to the dual drug base coating as may be readily seen from a comparison of FIGS. 16 and 13. In FIG. 16, curve 1602 represents the cilostazol and curve 1604 represents the sirolimus. However, the percentage release of both drugs were comparable at each time point. The respective in vitro release rate profiles are shown in FIG. 16, with the diamonds representing cilostazol and the squares representing sirolimus. In a comparison to the dual drug base coating, both drugs were released at a much faster rate, mirroring the fast release profiles shown in the in vivo PK study. Accordingly, combining the drugs in a single layer results in a higher degree of control over the elution rate.

The combination of a rapamycin, such as sirolimus, and cilostazol, as described above, may be more efficacious than either drug alone in reducing both smooth muscle cell proliferation and migration. In addition, as shown herein, cilostazol release from the combination coating may be controlled in a sustained fashion to achieve prolonged anti-platelet deposition and prevention of thrombosis formation on the stent surface or the surface of other blood contacting medical devices. The incorporation of cilostazol in the combination coating may be arranged in both a single layer with sirolimus or in a separate layer outside of the sirolimus containing layer. With its relatively low solubility in water, cilostazol has a potential to be retained in the coating for a relatively long period of time inside the body after deployment of the stent or other medical device. The relatively slow in vitro elution as compared to sirolimus in the inner layer suggests such a possibility. Cilostazol is stable, soluble in common organic solvents and is compatible with the various coating techniques described herein. It is also important to note that both sirolimus and cilostazol may be incorporated in a non-absorbable polymeric matrix or an absorbable matrix.

As illustrated above, the combination of sirolimus and cilostazol may be more efficacious than either drug alone in reducing both smooth muscle cell proliferation and migration. Cilostazol may also be utilized to achieve prolonged anti-platelet deposition and prevention of thrombosis on the stent or other medical device. In addition, cilostazol, which is a PDE-III active compound, also functions synergistically with selected adenosine A_{2A} receptor agonists described herein. Phosphodiesterase inhibitors act by blocking or retarding the enzymatic conversion of cyclic-adenosine monophosphate (c-AMP) to 5′ adenosine monophosphate (5′-AMP). Adenosine receptor agonist are G-protein coupled receptors that activate adenyl cyclase and elevate intracellular cAMP. In that sense, the combination of a PDEi and a selective adenosine receptor agonist delivered together will provide an enhanced level of stimulation of adenosine receptors compared to either class given alone. Accordingly, the combination of drugs may be utilized to treat restenosis, thrombosis, and to reduce myocardial injury following an acute myocardial infarction when delivered from a stent or other medical device.

A stent with through-hole reservoirs as described herein may be utilized to deliver one or more therapeutic agents for treating one or more conditions as described above. Multiple reservoirs may be utilized to deliver the one or more therapeutic agents luminally into the blood stream, abluminally into the tissue surrounding the device or both luminally and abluminally. For example, the rapamycin, either alone or in combination with the cilostazol may be delivered into the tissue surrounding the device to treat restenosis, thrombosis and inflammation. As described above, these therapeutic agents may potentiate each others actions in the treatment of these conditions. The cilostazol may also be configured for delivery into the blood stream alone or in combination with a selective adenosine A_{2A} receptor agonist to treat or reduce myocardial injury. In other words, a combination of a selective adenosine A2A agonist, a rapamycin such as sirolimus and a PDEi such as cilostazol may be utilized to increase adenosine levels in down stream tissue and to more effectively prevent restenosis and thrombosis formation.

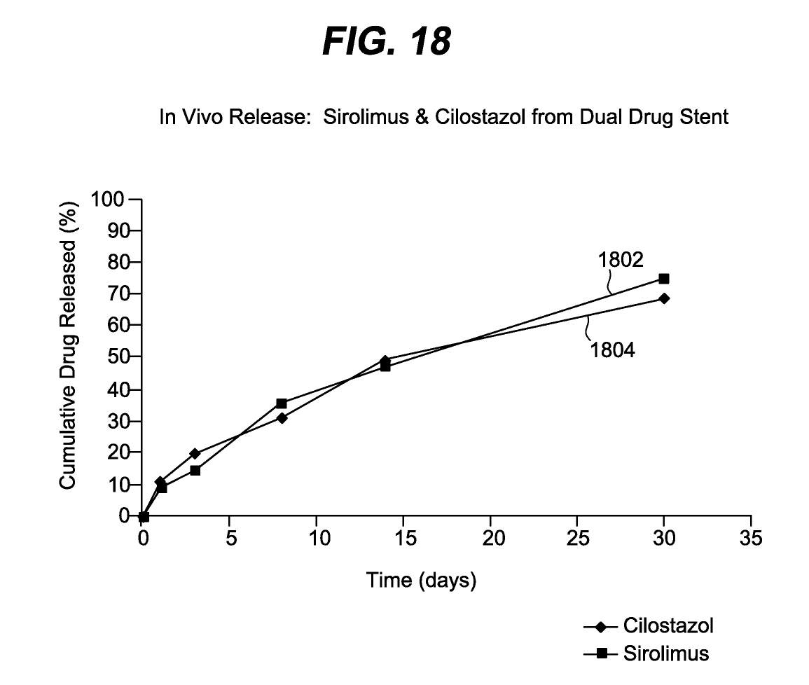

FIG. 18 graphically illustrates the release kinetic curves for sirolimus 1902 and cilostazol 1904 over a period of thirty (30) days from an in vivo release kinetics study. As illustrated, the release kinetics are similar for both drugs with approximately seventy (70) percent of both drugs being released from the stent at thirty (30) days (sirolimus being a little higher than the cilostazol). A 3.5×17 mm stent with reservoirs was utilized in the study. Each drug was placed individually in a reservoir to create an alternating pattern. The basic construct of each reservoir fill was a base (luminal side of the stent) comprising only polymer (PLGA_{75/25}) and no drug, a drug matrix either comprising 120.4 micro grams of cilostazol and 197.1 micro grams of polymer (PLGA_{75/25}) or 146.9 micro grams of sirolimus and 175.0 micro grams of polymer (PLGA_{75/25}), and a cap comprising only polymer (PLGA_{75/25}). The stent was placed in a standard porcine coronary artery model to evaluate the in vivo release kinetics and tissue concentration of both drugs released from the stent over time.

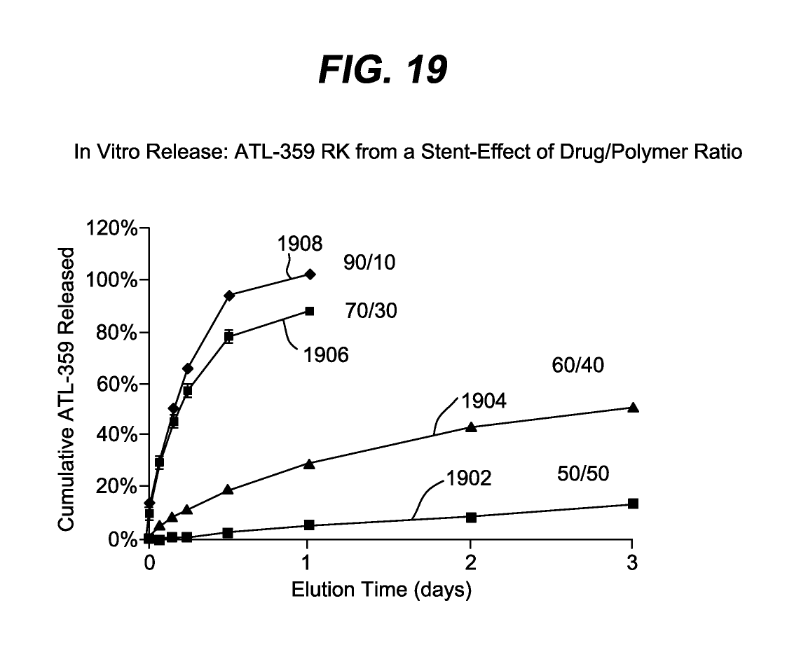

FIG. 19 graphically illustrates the release kinetic curves for ATL-359 over a period of six (6) days from an in vitro release kinetics study. This study was conducted using a 3.5×17 mm stent. A USP-7 apparatus was used to determine the drug release profile with a release media of phosphate buffered saline containing 4 percent by weight bovine serum albumin at 37 C. Curve 1902 illustrates the release kinetics for a drug (ATL-359) to polymer (PLGA_{75/25}) ratio or D/P ratio of 50/50. Curve 1904 illustrates the release kinetics for a drug (ATL-359) to polymer (PLGA75/25) ratio or D/P ratio of 60/40. Curve 1906 illustrates the release kinetics for a drug (ATL-359) to polymer (PLGA_{75/25}) ratio or D/P ratio of 70/30. Curve 1908 illustrates the release kinetics for a drug (ATL-359) to polymer (PLGA_{75/25}) ratio or D/P ratio of 90/10. As expected, the higher the D/P ratio, the faster the elution of the drug from the stent. Accordingly, by manipulating the drug to polymer ratio, one may adjust the release kinetics of the drug to accommodate the desired release profile discussed herein.

Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims.

What is claimed is:

1. A medical device for the local delivery of a selective adenosine receptor agonist in combination with at least one additional therapeutic agent for the treatment of myocardial injury following an acute myocardial infarction comprising:
an expandable intraluminal device configured for opening and reestablishing blood flow in a vessel at least partially occluded;
a selective adenosine receptor agonist releasably affixed to the expandable intraluminal device, the selective adenosine receptor agonist being configured to elute into the bloodstream at a rate of at least ten micrograms per hour for at least four (4) hours after the reestablishment of blood flow in the vessel; and
a phosphodiesterase inhibitor affixed to the expandable intraluminal device and configured to elute into at least one of the bloodstream and the surrounding tissue.

2. The medical device for the local delivery of a selective adenosine receptor agonist according to claim 1, wherein the expandable intraluminal device comprises a stent.

3. The medical device for the local delivery of a selective adenosine receptor agonist according to claim 2, wherein the stent comprises reservoirs.

4. The medical device for the local delivery of a selective adenosine receptor agonist according to claim 3, wherein the selective adenosine receptor agonist comprises an adenosine A_{2A} receptor agonist.

5. The medical device for the local delivery of a selective adenosine receptor agonist according to claim 4, wherein the phosphodiesterase inhibitor comprises cilostazol.

6. The medical device for the local delivery of a selective adenosine receptor agonist according to claim 5, wherein the adenosine A_{2A} receptor agonist is deposited into at least a first portion of the reservoirs and is configured for elution into the bloodstream.

7. The medical device for the local delivery of a selective adenosine receptor agonist according to claim 6, wherein the cilostazol is deposited into at least a second portion of the reservoirs and configured for elution into at least one of the blood stream and the surrounding tissue.

8. The medical device for the local delivery of a selective adenosine receptor agonist according to claim 7, further comprising an anti-restenotic agent.

9. The medical device for the local delivery of a selective adenosine receptor agonist according to claim 8, wherein the anti-restenotic agent comprises a rapamycin.

10. The medical device for the local delivery of a selective adenosine receptor agonist according to claim 9, wherein the rapamycin is deposited into at least a third portion of the reservoirs and is configured to elute into the surrounding tissue.

11. The medical device for the local delivery of a selective adenosine receptor agonist according to claim 10, wherein the adenosine A_{2A} receptor agonist is mixed with a polymer.

12. The medical device for the local delivery of a selective adenosine receptor agonist according to claim 11, wherein the cilostazol is mixed with a polymer.

13. The medical device for the local delivery of a selective adenosine receptor agonist according to claim 12, wherein the rapamycin is mixed with a polymer.

14. A method for treating myocardial injury following an acute myocardial infarction comprising:
expanding an intraluminal device to open and reestablish blood flow in a vessel at least partially occluded;
releasing a selective adenosine receptor agonist from the expandable intraluminal device into the bloodstream at a rate of at least ten micrograms per hour for at least four (4) hours after the reestablishment of blood flow in the vessel; and
releasing a phosphodiesterase inhibitor from the expandable intraluminal device into at least one of the bloodstream and the surrounding tissue.

15. The method for treating myocardial injury according to claim 14, further comprising releasing a rapamycin from the expandable intraluminal device into the tissue surrounding the device.
